(19) United States
(12) Patent Application Publication
(10) Pub. No.: US 2025/0345465 A1
(43) Pub. Date: Nov. 13, 2025

(54) DUAL T1/T2 MRI CONTRAST AGENTS FOR PHOTOTHERMAL THERAPY

(71) Applicant: William Marsh Rice University, Houston, TX (US)

(72) Inventors: Naomi J. Halas, Houston, TX (US); Yara Kadria-Vili, Houston, TX (US); Oara Neumann, Houston, TX (US)

(73) Assignee: William Marsh Rice University, Houston, TX (US)

(21) Appl. No.: 18/859,947

(22) PCT Filed: Apr. 25, 2023
(86) PCT No.: PCT/US2023/019749

§ 371 Date: Oct. 24, 2024

Related U.S. Application Data
(60) Provisional application No. 63/334,622, filed on Apr. 25, 2022.

(51) Int. Cl. A61K 49/18; A61K 41/00

(52) U.S. Cl. CPC A61K 49/183; A61K 41/0052

(57) ABSTRACT

A photothermal magnetic resonance imaging enhancement agent includes composite nanoparticles. The composite nanoparticle includes an inner layer of a dielectric material with a porous substrate having pores, an inner layer with a core, magnetically responsive nanoparticles disposed on the porous substrate, and an outer layer of a metallic material around the inner layer and the magnetically responsive nanoparticles. A method of making a photothermal magnetic resonance imaging enhancement agent includes synthesizing a dielectric substrate, baking the dielectric substrate to generate pores, synthesizing magnetically responsive nanoparticles, loading the magnetically responsive nanoparticles into the pores, attaching linker molecules to the dielectric core, attaching a metal nanoparticle to at least a portion of the linker molecules, reducing additional metal onto the metal nanoparticles to form an outer layer disposed on the dielectric inner layer, and selecting reducing a condition such that the outer layer has a controllable thickness forming a composite nanoparticle.

### CROSS-REFERENCE TO RELATED APPLICATION

This International Patent application claims priority from U.S. Provisional Application No. 63/334,622, filed on Apr. 25, 2022. The content of this application is hereby incorporated by reference herein in its entirety.

### STATEMENT REGARDING FEDERALLY SPONSORED RESEARCH

This invention was made with government support under Grant No. T32CA196561 awarded by the National Institutes of Health. The government has certain rights in the invention.

### BACKGROUND

A variety of nanoparticles (NPs) have been developed for photothermal therapy PTT, including metallic NPs, carbon-based NPs, and organic/inorganic nanohybrid NPs.

Nanoparticles with optical properties in the near-infrared (NIR) (at wavelengths where tissue is highly transparent and ˜3 cm penetration depth is possible) have opened a new avenue to photothermally treat solid tumors. NIR illumination at the nanoparticle plasmon resonance induces collective oscillations of the nanoparticle conduction band electrons, causing an increase in local temperature that can initiate various cell death pathways. Thus, there is interest in methods of monitoring the NPs at a tumor site during the photothermal treatment. This invention was funded in part by the Robert A. Welch Foundation under Welch Grant No. C-1220.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, one or more embodiments disclosed herein relate to a photothermal magnetic resonance imaging (MRI) enhancement agent. The photothermal magnetic resonance imaging enhancement agent includes a plurality of composite nanoparticles, each composite nanoparticle includes a dielectric inner layer with a porous substrate having pores, a plurality of magnetically responsive nanoparticles disposed on the porous substrate, and a metallic outer layer around the inner layer and the magnetically responsive nanoparticles. In one or more embodiments, when the inner layer is a core, the core includes a dielectric material. In one or more embodiments of the present disclosure, the inner layer is disposed around a core, such that the composite nanoparticles discussed herein include the core, the inner layer, one or more magnetically responsive nanoparticles, and an outer layer. In one or more embodiments, when the inner layer is disposed around a core, the core includes a metallic material. The metallic material in the core may be the same or different than the metallic material in the outer layer.

In one or more embodiments, an average outer diameter of the inner layer is between about 80 nm and about 110 nm.

In one or more embodiments, the inner layer includes a dielectric core.

In one or more embodiments, the inner layer includes a dielectric layer disposed around a metallic core.

In one or more embodiments, the magnetically responsive nanoparticles have an average diameter between about 2 nm and about 3 nm.

In one or more embodiments, the magnetically responsive nanoparticles include gadolinium oxide.

In one or more embodiments, the composite nanoparticle is selected from the group consisting of a type 1 (T₁) contrast agent and a type 2 (T₂) contrast agent.

In one or more embodiments, the composite nanoparticle has a relaxivity rate r₁ of at least 3.6 times greater than a reference gadopentetate dimeglumine T₁ MRI contrast agent.

In one or more embodiments, the composite nanoparticle has a relaxivity rate r₂ comparable to a reference superparamagnetic iron oxide T₂ MRI contrast agent.

In one or more embodiments, the composite nanoparticle is a type 1 contrast agent and a type 2 contrast agent.

In one or more embodiments, a surface area of the porous substrate is between about 900 m²/g to about 1000 m²/g.

In one or more embodiments, an average pore diameter of the pores is between about 1.5 nm and about 4 nm.

In one or more embodiments, the porous substrate includes a dielectric material selected from the group consisting of silicon dioxide, titanium dioxide, PMMA, polystyrene, dendrimers, and combinations thereof.

In one or more embodiments, the porous substrate includes mesoporous silica.

In one or more embodiments, the metallic material includes a metal selected from the group consisting of coinage metals, noble metals, transition metals, and synthetic metals.

In one or more embodiments, the metal includes gold.

In one or more embodiments, the metallic material includes a metal shell with an average thickness of about 10 nm to about 30 nm.

In one or more embodiments, the composite nanoparticle further includes a coating surrounding the metallic material, wherein the coating includes molecules that allow one or more of improved nanoparticle stability, facilitating bypassing of an immune system, targeting cells, and increased circulation time.

In one or more embodiments, the composite nanoparticle has a surface plasmon resonance between about 800 nm to about 1100 nm.

In one or more embodiments, the composite nanoparticle induces a temperature increase of about 20 to about 55° C. upon irradiation with a NIR laser at a laser power of between about 1 W to about 5 W.

In another aspect, one or more embodiments disclosed herein relate to a method of making a photothermal magnetic resonance imaging enhancement agent. The method includes synthesizing a dielectric substrate, baking the dielectric substrate to generate pores within the dielectric substrate, synthesizing magnetically responsive nanoparticles, loading the magnetically responsive nanoparticles into the pores of the dielectric substrate so as to form a dielectric inner layer comprising the dielectric substrate and the magnetically responsive nanoparticles, attaching a plurality of linker molecules to the dielectric core, attaching a metal nanoparticle to each of at least a portion of the linker molecules, reducing additional metal onto the metal nanoparticles so as to form an outer layer disposed on the dielectric inner layer, and selecting a condition of the reducing such that the outer layer has a controllable thickness forming a composite nanoparticle.

In yet another aspect, one or more embodiments describe a system for visualizing and inducing hyperthermia in a cell or tissue comprising the steps of synthesizing composite nanoparticles, delivering the composite nanoparticles to the cell or tissue, visualizing the composite nanoparticles to ensure site specific delivery, and exposing the composite nanoparticles to infrared radiation under conditions where the composite nanoparticles emit heat upon exposure to the infrared radiation.

Other aspects and advantages of the claimed subject matter will be apparent from the following description and the appended claims.

### BRIEF DESCRIPTION OF DRAWINGS

FIG. 1A shows a of cross-sectional schematic of a composite nanoparticle in accordance with one or more embodiments;

FIG. 1B shows a of cross-sectional schematic of a composite nanoparticle in accordance with one or more embodiments;

FIG. 2 shows a of cross-sectional schematic of a composite nanoparticle in accordance with one or more embodiments;

FIG. 3A shows an exemplary transmission electron microscope (TEM) image of a plurality of non-conducting dielectric inner layers formed during the synthesis of a plurality of composite nanoparticles in accordance with one or more embodiments where the scale bar indicates 50 nm;

FIG. 3B shows an exemplary transmission electron microscope (TEM) image of a plurality of magnetically responsive nanoparticles formed during the synthesis of a plurality of composite nanoparticles, where the scale bar indicates 20 nm;

FIG. 3C shows an exemplary histogram plot of the diameter distribution of a plurality of Gd₂O₃-mesoporous silica nanoparticles in accordance with one or more embodiments;

FIG. 3D shows an exemplary transmission electron microscope (TEM) image of a composite nanoparticle in accordance with one or more embodiments, where the scale bar indicates 50 nm;

FIG. 4A shows exemplary extinction spectra of a plurality of composite nanoparticles as a function of outer layer thickness in accordance with one or more embodiments;

FIG. 4B shows an exemplary plot the outer layer thickness distribution determined by high-resolution transmission electron microscopy of a plurality of composite nanoparticles in accordance with one or more embodiments;

FIG. 4C shows an exemplary plot the outer layer thickness distribution determined by high-resolution transmission electron microscopy of a plurality of composite nanoparticles in accordance with one or more embodiments;

FIG. 4D shows an exemplary plot the outer layer thickness distribution determined by high-resolution transmission electron microscopy of a plurality of composite nanoparticles in accordance with one or more embodiments;

FIG. 5A shows exemplary plots of longitudinal magnetization recovery (T₁) vs. recovery time at various Gd³⁺ concentrations of composite nanoparticles in water in accordance with one or more embodiments;

FIG. 5B shows exemplary plots of the decay of transverse magnetization recovery (T₂) vs. echo time at various Gd³⁺ concentrations of composite nanoparticles in water in accordance with one or more embodiments;

FIG. 5C shows an exemplary image of the T₁-weighted (T_{1w}) enhancement at various Gd³⁺ concentrations of composite nanoparticles in water in accordance with one or more embodiments;

FIG. 5D shows an exemplary image of the T₂-weighted (T_{2w}) enhancement at various Gd³⁺ concentrations of composite nanoparticles in water in accordance with one or more embodiments;

FIG. 5E shows an exemplary plot of the R₁ and R₂ relaxivity as a function of Gd(III) ion concentration in composite nanoparticles in water in accordance with one or more embodiments;

FIG. 5F shows an exemplary plot of the r₁ and r₂ relaxivity rates and their ratio r₂/r₁ of a plurality of composite nanoparticles with different outer layer thickness, magnetically responsive nanoparticles, dielectric inner layers comprising a porous substrate with magnetically responsive nanoparticles in the pores and attached metal nanoparticles, and comparative MRI contrast agents in water in accordance with one or more embodiments;

FIG. 5G shows an exemplary plot of the enhancement factor of a plurality of particles of a plurality of composite nanoparticles with different outer layer thickness, magnetically responsive nanoparticles, dielectric inner layers comprising a porous substrate with magnetically responsive nanoparticles in the pores and attached metal nanoparticles, and comparative MRI contrast agents relative to a standard T₁ MRI contrast agent in water in accordance with one or more embodiments;

FIG. 5H shows an exemplary plot of r₁ relaxivity rate values versus gold outer layer thickness in water in accordance with one or more embodiments;

FIG. 6A shows exemplary plots of longitudinal magnetization recovery (T₁) vs. recovery time at different concentrations of composite nanoparticles in 0.48% agarose phantoms in accordance with one or more embodiments;

FIG. 6B shows exemplary plots of decay of transverse magnetization recovery (T₂) vs. echo time at different concentrations of composite nanoparticles in 0.48% agarose phantoms in accordance with one or more embodiments;

FIG. 6C shows an exemplary plot of the R₁ and R₂ relaxivity as a function of Gd(III) ion concentration in composite nanoparticles in 0.48% agarose phantoms in accordance with one or more embodiments;

FIG. 6D shows an exemplary T₁ map values of composite nanoparticles at different concentrations in 0.48% agarose phantoms in accordance with one or more embodiments;

FIG. 6E shows an exemplary T₂ map values of composite nanoparticles at different concentrations in 0.48% agarose phantoms in accordance with one or more embodiments;

FIG. 7A shows exemplary plots of longitudinal magnetization recovery (T₁) vs. recovery time of different concentrations of composite nanoparticles in 0.48% agarose phantoms in accordance with one or more embodiments;

FIG. 7B shows exemplary plots of the decay of transverse magnetization recovery (T₂) vs. echo time of different concentrations of composite nanoparticles in 0.48% agarose phantoms in accordance with one or more embodiments;

FIG. 7C show exemplary T_{2w} MR images of different concentrations of composite nanoparticles in 0.48% agarose phantoms at different echo times according to one or more embodiments;

FIG. 7D shows an exemplary plot of the signal intensities of the T_{2w} MR images in FIG. 5C according to one or more embodiments;

FIG. 7E shows exemplary images of MR signal acquired in 0.48% agarose phantoms according to one or more embodiments;

FIG. 7F shows an exemplary plot of MRI signal intensities relative to water MR images in FIG. 5E according to one or more embodiments;

FIG. 8A shows a schematic representation of the MRI-guided photothermal illumination setup in accordance with one or more embodiments;

FIG. 8B shows an exemplary T₁ weighted MR image obtained before PTT in accordance with one or more embodiments;

FIG. 9A shows an exemplary plot of thermal MRI mapping showing the temperature changes of different concentrations of composite nanoparticles in 0.48% agarose phantoms in accordance with one or more embodiments;

FIG. 9B shows an exemplary plot of the temperature change during photothermal treatment at one concentration of composite nanoparticles in 0.48% agarose phantoms in accordance with one or more embodiments;

FIG. 9C shows an exemplary plot of the temperature change during photothermal treatment at different concentrations of composite nanoparticles in 0.48% agarose phantoms in accordance with one or more embodiments; and

FIG. 9D shows an exemplary plot of the temperature change during photothermal treatment at different concentrations of composite nanoparticles in 0.48% agarose phantoms in accordance with one or more embodiments.

### DETAILED DESCRIPTION

Specific embodiments will now be described in detail with reference to the accompanying figures. In the following description, numerous details are set forth to provide an understanding of the present disclosure. However, it will be understood by those skilled in the art that embodiments of the present disclosure may be practiced without these details and that numerous variations or modifications from the described embodiments may be possible.

In general, one or more embodiments of the present disclosure relate to a photothermal magnetic resonance imaging contrast enhancement agent comprising a plurality of composite nanoparticles with properties to enhance magnetic resonance imaging and/or photothermal ablation. Further, in one or more embodiments of the present disclosure the composite nanoparticles may be functionalized with a polymer, for example polyethylene glycol-based polymer. The polymer may be present in a coating.

One or more embodiments of the present disclosure may combine the composite nanoparticles with an antibody and/or peptide targeting and/or therapeutic actuation. The antibody may be present in a coating. An exemplary antibody is a folate receptor adapted for targeting cancer cells.

In one or more embodiments of the present disclosure, antibody targeting may be used such that the composite nanoparticles may bind to the surface receptors of specific cell types. In the case of cancer therapy, the composite nanoparticles may allow for the tracking the location of the particles in vivo. For example, photothermal magnetic resonance imaging may be used to follow the path of the particles or verify the quantity of particles at specific locations. Once verified, ablation of the targeted cells may be carried out by photothermal ablation. Further, in one or more embodiments the composite nanoparticles described herein may be used for one or more of a variety of imaging application and light induced drug release of therapeutic molecules.

Additionally, one or more embodiments of the present disclosure relate to methods, devices, materials, and/or systems including composite nanoparticles. The composite nanoparticles may be used in hyperthermia in a cell or tissue. The composite nanoparticles may enable imaging, targeted drug delivery, and photothermal therapy to be conducted. Further, the composite nanoparticles may be used to perform other processes without departing from the invention.

In one or more embodiments of the present disclosure, each composite nanoparticle or a portion of the composite nanoparticles may be one or more of photothermal-active and magnetically responsive, e.g., generate photothermal response and/or MRI contrast when illuminated and/or imaged using an appropriate technique.

In one or more embodiments of the present disclosure, the photothermal magnetic resonance contrast enhancement agent may be a dual type 1 (T₁) and type (T₂) contrast agent.

In one or more embodiments of the present disclosure, the composite nanoparticles may have a photothermal response upon NIR laser illumination. The photothermal response of the composite nanoparticle may be used for photothermal therapy. In one or more embodiments, the composite nanoparticle induces a temperature increase of about 20 to about 55° C. upon irradiation with a NIR laser at a laser power of between about 1 W to about 5 W.

### Composite Nanoparticles

In one or more embodiments of the present disclosure, the composite nanoparticles discussed herein include an inner layer, one or more magnetically responsive nanoparticles, and an outer layer around the inner layer and the magnetically responsive nanoparticles. In one or more embodiments, the outer layer is a shell around a core. The inner layer may be the core. Alternatively, the inner layer maybe disposed around the core. In one or more embodiments, the outer layer includes a metallic material.

In one or more embodiments of the present disclosure, the composite nanoparticles discussed herein the inner layer is a core such that the composite nanoparticles include the core, one or more magnetically responsive nanoparticles, and an outer layer. In one or more embodiments, when the inner layer is a core, the core includes a dielectric material. In one or more embodiments of the present disclosure, the inner layer is disposed around a core, such that the composite nanoparticles discussed herein include the core, the inner layer, one or more magnetically responsive nanoparticles, and an outer layer. In one or more embodiments, when the inner layer is disposed around a core, the core includes a metallic material. The metallic material in the core may be the same or different than the metallic material in the outer layer.

In one or more embodiments of the present disclosure, the inner layer is a non-conducting dielectric material. In one or more embodiments the dielectric material is selected from the group consisting of silicon dioxide, titanium dioxide, PMMA, polystyrene, dendrimers, and combinations thereof. However, the inner layer may be a different dielectric material than those listed above without departing from the present disclosure. In one or more embodiments of the present disclosure, the inner layer is mesoporous silica (SiO₂).

In accordance with one or more embodiments of the present disclosure, the average outer diameter of the inner layer is between about 70 nm to about 150 nm. The average outer diameter of the inner layer in one or more embodiments may have a lower limit of one of 70 nm, 75 nm, 80 nm, 85 nm, and 90 nm and an upper limit of one of 95 nm, 100 nm, 105 nm, 110 nm, 115 nm, 120 nm, 125 nm, 130 nm, 135 nm, 140 nm, 145 nm, and 150 nm.

In one or more embodiments of the present disclosure, the inner layer is a porous substrate having pores. The pores may have an average pore size of about 1.5 nm to about 4 nm in one or more embodiments. The average outer diameter of the inner layer in one or more embodiments may have a lower limit of one of 1.5 nm, 1.6 nm, 1.7 nm, 1.8 nm, 1.9 nm, 2.0 nm, 2.1 nm, 2.2 nm, 2.3 nm, 2.4 nm, and 2.5 nm and an upper limit of one of 2.6 nm, 2.7 nm, 2.8 nm, 2.9 nm, 3.0 nm, 3.1 nm, 3.2 nm, 3.3 nm, 3.4 nm, 3.5 nm, 3.6 nm, 3.7 nm, 3.8 nm, 3.9 nm and 4.0 nm.

In one or more embodiments of the present disclosure, the surface area of the porous substrate is between about 900 m²/g to about 1000 m²/g. The average surface area of the porous substrate in one or more embodiments may have a lower limit of one of 900 m²/g, 905 m²/g, 910 m²/g, 905 m²/g, 920 m²/g, 925 m²/g, 930 m²/g, 935 m²/g, and 940 m²/g and an upper limit of one of 945 m²/g, 950 m²/g, 955 m²/g, 960 m²/g, 965 m²/g, 970 m²/g, 975 m²/g, 980 m²/g, 985 m²/g, 990 m²/g, 995 m²/g, and 1000 m²/g.

In one or more embodiments of the present disclosure, the pores house at least one magnetically responsive nanoparticle. In one or more embodiments, the magnetically responsive nanoparticles may be paramagnetic or ferromagnetic. In one or more embodiments of the present disclosure, the magnetically responsive nanoparticle is a dual T₁/T₂ MRI contrast agent.

In one or more embodiments of the present disclosure, the magnetically responsive nanoparticle is a transition metal and/or a rare earth metal. In one or more embodiments of the present disclosure, the transition metal and/or a lanthanide are selected from gadolinium (III), iron (II), iron (III) and/or manganese (II). The magnetically responsive nanoparticle may include any number, type, and/or combination of metal ions without departing from the disclosure. In one or more embodiments of the present disclosure, the magnetically responsive nanoparticle may be gadolinium oxide (Gd₂O₃). In yet another embodiment, the Gd(III) may include any Gd(III) organic framework.

In one or more embodiments of the present disclosure, the magnetically responsive nanoparticle has an average hydrodynamic diameter of about 2 nm to about 4 nm. The average hydrodynamic diameter of the magnetically responsive nanoparticle in one or more embodiments may have a lower limit of one of 2.0 nm, 2.1 nm, 2.2 nm, 2.3 nm, 2.4 nm, 2.5 nm, 2.6 nm, 2.7 nm, 2.8 nm, 2.9 nm, and 3.0 nm and an upper limit of one of 3.1 nm, 3.2 nm, 3.3 nm, 3.4 nm, 3.5 nm, 3.6 nm, 3.7 nm, 3.8 nm, 3.9 nm and 4.0 nm.

In one or more embodiments, magnetically responsive nanoparticles are disposed on the inner layer. The magnetically responsive nanoparticles may be disposed in the pores of the inner layer. The magnetically responsive nanoparticles in the pores may be disposed on the surfaces of the pores of the dielectric core. Thus, magnetically responsive nanoparticles in the pores may also be on the inner layer. The magnetically responding nanoparticles may be disposed in pores in an outer portion of the inner layer. In one or more embodiments, the outer portion of the inner layer is the exterior surface of the inner layer. In one or more embodiments, the outer portion of the inner layer is a surface region of the inner layer. The magnetically response nanoparticles may be additionally disposed in pores in an interior portion of the inner layer. In one or more embodiments, the interior portion of the inner layer is the bulk of the inner layer. In one or more embodiments, the outer and interior portions combine to form the whole of the inner layer. In one or more embodiments, 50% of the magnetically responsive nanoparticles may be distributed in an outer portion of the inner layer and 50% may be uniformly distributed in the bulk of the inner layer. The magnetically responsive nanoparticles may be doped with a linker molecule. The linker molecule may facilitate the attachment of the magnetically responsive nanoparticles to the inner layer. Linker molecules may include but are not limited to amino silanes, carboxy silanes, or hydroxy silanes.

In one or more embodiments of the present disclosure, the outer layer may be disposed around the inner layer and the magnetically responsive nanoparticles. The outer layer may encapsulate the dielectric core and the magnetically responsive nanoparticles. The outer layer may be a metallic material. The metallic material may be, for example, coinage metals, noble metals, transition metals, and synthetic metals. However, the outer layer may be a different metallic material than those listed above without departing from the present disclosure. In one or more embodiments the outer layer may be gold.

In one or more embodiments, the outer layer may have other materials disposed on an exterior side of the outer layer. For example, polymeric, ceramic, targeting molecules, fluorescing material, other MRI-contrast agents or other materials may be disposed on an exterior surface of the outer layer. In one or more embodiments of the present disclosure, the outer layer may include a fluorescing material. For example, the fluorescing material may be attached or chemically linked to the outer layer. The fluorescing material may be, for example, a fluorescing dye. However, other fluorescing materials may be used without departing from the present. In one or more embodiments of the invention, the exterior of the outer layer may be functionalized with molecules including polyethylene glycol (PEG), DNA/aptamers, proteins, polypeptides, antibodies, or other polymeric molecules.

In one or more embodiments, the thickness of the outer layer may be from about 10 nm to about 50 nm. The thickness of the outer layer in one or more embodiments may have a lower limit of one of 10 nm, 11 nm, 12 nm, 13 nm, 14 nm, 15 nm, 16 nm, 17 nm, 18 nm, 19 nm, 20.0 nm, 21 nm, 22 nm, 23 nm, 24 nm, 25 nm, 26 nm, 27 nm, 28 nm, 29 nm, 30 nm, 31 nm, 32 nm, 33 nm, 34 nm, and 35 nm, and an upper limit of one of 36 nm, 37 nm, 38 nm, 39 nm, 40 nm, 41 nm, 42 nm, 43 nm, 44 nm, 45 nm, 46 nm, 47 nm, 48 nm, 49 nm, and 50 nm.

The thickness of the outer layer may allow for tailoring photothermal magnetic resonance imaging enhancement agent to have a plasmon resonance that is tuned to the near-IR window of the electromagnetic spectrum (i.e., from about 700 nm to 2500 nm). Near-infrared light can penetrate biological tissues more efficiently than visible light because tissue scatters and absorbs less light at the longer NIR wavelengths. In one or more embodiments, the particles according to the present disclosure may have a plasmon resonance that peaks in a region between about 800 nm and 1350 nm. These particular wavelengths may be particularly preferred for in vivo imaging because they can improve signal-to-noise ratios by reducing background noise caused by tissue.

The composite nanoparticles according to the present disclosure may have a plasmon resonance that peaks in a region between about 800 nm to about 1350 nm or about 800 nm to about 1100 nm or about 800 nm to about 1000 nm or about 800 nm to about 900 nm.

In one or more embodiments of the present disclosure, the outer layer may contain a monolayer of a polymer. The polymer in one or more embodiments may be a polyethylene glycol-based polymer. The polyethylene glycol-based polymer monolayer may improve composite nanoparticle stability, facilitate the bypassing of the immune system, and increase circulation time in in vivo studies.

In one or more embodiments of the present disclosure, the molecular weight of the polyethylene glycol based-polymer may be between about 1000 Da to about 5000 Da. The molecular weight of the polyethylene glycol based-polymer in one or more embodiments may have a lower limit of one of 1000 Da, 1500 Da, 2000 Da, and 2500 Da and an upper limit of one of 3000 Da, 3500 Da, 4000 Da, 4500 Da, and 5000 Da.

In one or more embodiments of the present disclosure, the polyethylene glycol based-polymer is methoxy-polyethylene glycol thiol.

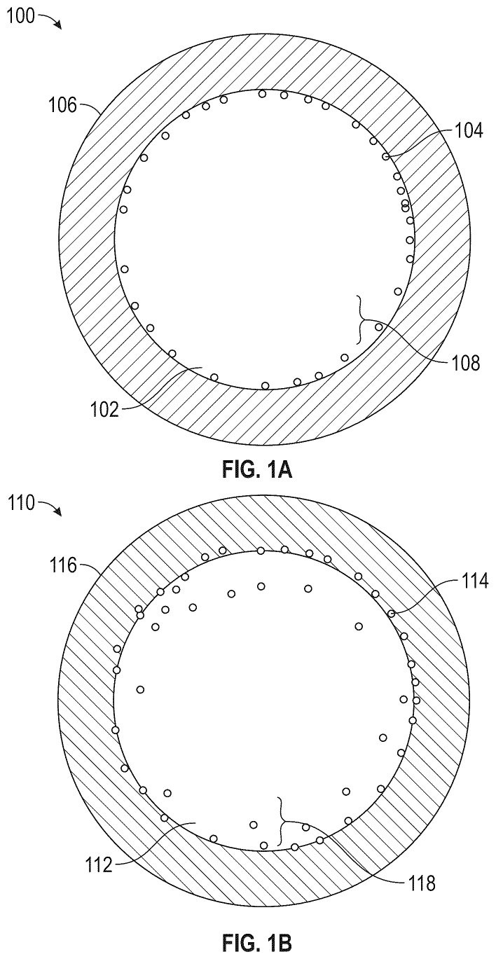

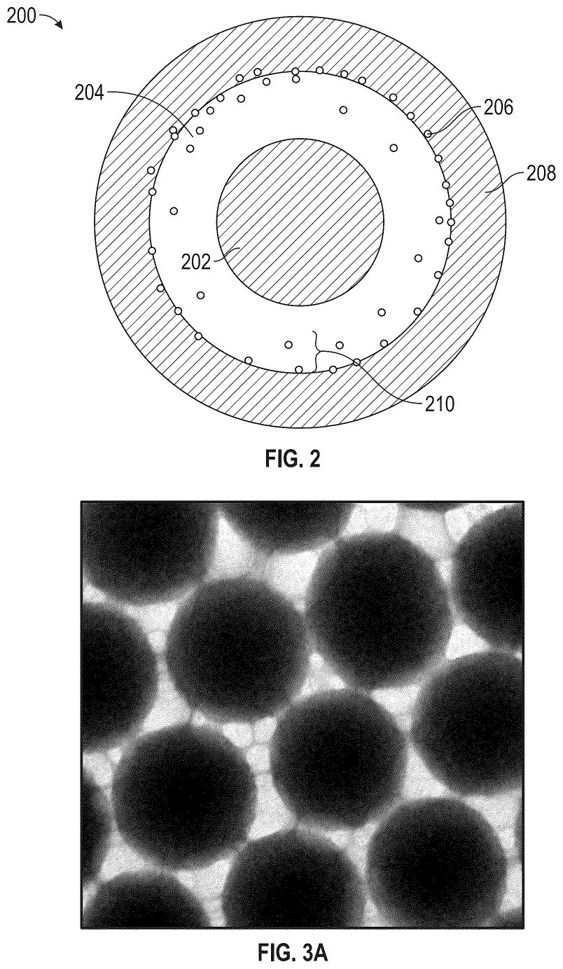

In accordance with one or more embodiments of the present disclosure, FIG. 1A illustrates a schematic representation of a cross-section of a composite nanoparticle (100). The particle may include a dielectric inner layer (102), magnetically responsive nanoparticles (104) attached to the dielectric inner layer (102), and a metallic outer layer (106). The dielectric inner layer includes a porous substrate. The porous substrate extends throughout an outer region (108).

In accordance with one or more embodiments of the present disclosure, FIG. 1B illustrates a schematic representation of a cross-section of a composite nanoparticle (110). The particle may include a dielectric inner layer (112), magnetically responsive nanoparticles (114) attached to the dielectric inner layer (112), and a metallic outer layer (116). The dielectric inner layer includes a porous substrate. The porous substrate extends throughout an outer region (118).

In accordance with one or more embodiments of the present disclosure, FIG. 2 illustrates a schematic representation of a cross-section of a composite nanoparticle (200). The particle may include a metal core (202), a dielectric inner layer (204), magnetically responsive nanoparticles (206) attached to the dielectric inner layer (204), and a metallic outer layer (208). The dielectric inner layer includes a porous substrate. The porous substrate extends throughout an outer region (210).

### Properties of Composite Nanoparticles

In magnetic resonance imaging (MRI), there are two types of contrast agents: type 1 (T₁) and type 2 (T₂). T₁ agents are positive contrast agents that make an image brighter on MRI phantoms. T₂ agents are negative contrast agents that cause a darker image on MRI phantoms. Contrast agents for MRI lighten or darken MRI phantoms by modifying the relaxation time of the spins of protons in water. Commercial T₁ contrast agents tend to need to be in direct contact with water to produce its effect while T₂ agents tend not to need to be in direct contact with water. Thermal magnetic resonance imaging (MRI) mapping can correlate thermal damage with the extent of thermal necrosis and enable real-time temperature evaluation during PTT.

In one or more embodiments, the magnetically responsive nanoparticle is a dual type 1/type 2 MRI contrast agent. Composite nanoparticles of the present disclosure may be dual T₁/T₂ MRI contrast agents and have a strong plasmon resonance in the NIR region where tissue is highly transparent (known as the first NIR therapeutic window).

Additional contrast approaches may be possible as follows. The MRI contrast signal of the present composite nanoparticles may be further enhanced by processing the ratio of T_{1w}/T_{2w} signal intensities. The present composite nanoparticles may boost the contrast in the processed MR image of T_{1w}/T_{2w} signal intensity ratio and may facilitate real-time temperature feedback under thermal MRI mapping, and may enhance PTT efficacy in solid tumors.

In accordance to one or more embodiments, the dual T₁ and T₂ MRI properties enhance MRI visualization in T₁ weighted (T_{1w}) MRI and T₂ weighted (T_{2w}) MRI.

The present composite nanoparticles may be used for MRI-guided localized NIR-photothermal therapy. The present composite nanoparticles at concentrations equivalent to current therapeutic doses 10⁹ composite nanoparticle/mL in agarose phantoms may provide sufficient contrast in MRI to enable localized photothermal heating under NIR illumination and may facilitate real-time temperature feedback through thermal MRI mapping.

In one or more embodiments, incorporating magnetically responsive nanoparticles within the dielectric porous substrate and encapsulating with a metal shell improves the r₁ and r₂ relaxivity values of magnetically responsive nanoparticles by at least 1 and 4 times respectively. This increase may be due to the decrease in the tumbling rate of the composite nanoparticle (τ_{R}) and the increase in the water exchange rate (τₘ) caused by the confinement space the channels and the pores within the structure were creating. Forming a continuous metal shell and increasing the metal shell thickness may decrease the relaxivity rates similarly as predicted by Solomon-Bloembergen-Morgan (SBM) theory.

In one or more embodiments, the present composite nanoparticles may have a relaxivity rate, r₁, at least 3 times and up to 4 times greater than a reference gadopentetate dimeglumine T₁ MRI contrast agent. For example, the reference gadopentetate dimeglumine T₁ MRI contrast agent may be Magnevist®, commercially available from Bayer.

In one or more embodiments, the present composite nanoparticles may have a relaxivity rate, r₂, comparable to a reference superparamagnetic iron oxide T₂ MRI contrast agent.

In one or more embodiments, a composite nanoparticle concentration of about 1.0×10⁹ composite nanoparticles/mL to about 9.0×10⁹ composite nanoparticles/mL causes a temperature of between 15° C. to 60° C. in agarose phantoms. The composite nanoparticle concentration in one or more embodiments is at least 1.0×10⁹ nanoparticles/mL, 1.5×10⁹ nanoparticles/mL, 2.0×10⁹ nanoparticles/mL, 2.5×10⁹ nanoparticles/mL, 3.0×10⁹ nanoparticles/mL, 3.5×10⁹ nanoparticles/mL, 4.0×10⁹ nanoparticles/mL, and 4.5×10⁹ nanoparticles/mL, and up to 5.0×10⁹ nanoparticles/mL, 5.5×10⁹ nanoparticles/mL, 6.0×10⁹ nanoparticles/mL, 6.5×10⁹ nanoparticles/mL, 7.0×10⁹ nanoparticles/mL, 7.5×10⁹ nanoparticles/mL, 8.0×10⁹ nanoparticles/mL, 8.5×10⁹ nanoparticles/mL, and 9.0×10⁹ nanoparticles/mL. The temperature increase in one or more embodiments may be at least 15° C., 20° C., 25° C., 30° C., and 35° C., and up to 45° C., 45° C., 50° C., 55° C., and 60° C.

### Composite Nanoparticle Synthesis

One or more embodiments of the present disclosure relate to method of synthesizing a photothermal magnetic resonance imaging enhancement agent. In accordance with one or more embodiments of the present disclosure, photothermal magnetic resonance imaging enhancement agent comprises a plurality of composite nanoparticles.

The method disclosed herein may include the synthesis of a dielectric core, magnetically responsive nanoparticles, and colloidal metal nanoparticles by wet chemical methods. The dielectric core may be transformed into a dielectric core including a porous substrate having pores by a baking process. In one or more embodiments, the dielectric core is the porous substrate. A magnetically responsive nanoparticle may be deposited onto the pore of the porous substrate by a sonication. In one or more embodiments, the dielectric core loaded with magnetically responsive nanoparticle may be functionalized with a linker molecule. The linker molecule may enable the attachment of colloidal metal nanoparticles. Following the deposition of the colloidal metal nanoparticles, an electroless plating process may be used to reduce more metal onto the dielectric core, forming a continuous metal shell. Lastly, the metal shell may be functionalized with a polyethylene-based polymer.

In one or more embodiments of the present disclosure, photothermal magnetic resonance imaging contrast enhancement agent is produced using a four step process including coating gold or other core material particles with APTES-doped dielectric, loading water and Gadolinium or other contrast material into the APTES-doped dielectric inner layer, etching the dielectric inner layer and seeding the dielectric inner layer with gold, and coating the dielectric inner layer with an outer layer of gold.

### EXAMPLES

The following examples are merely illustrative and should not be interpreted as limiting the scope of the present disclosure.

### Materials

200 proof ethanol was purchased from Decon Laboratories, Inc. Cetyltrimethylammonium chloride (CTAC) was purchased from Sisco Research Laboratories Pvt. Ltd. Ammonium hydroxide solution (28% NH₃ in water), Tetraethyl orthosilicate (TEOS, ≥99.9%), acetone (≥99.5%), gadolinium (III) chloride hexahydrate (GdCl₃·6H₂O, ≥99%), diethylene glycol (DEG, 99%), (3-aminopropyl)-triethoxysilane (APTES, 99%), sodium chloride (NaCl, ≥99%), and Tetrakis(hydroxymethyl) phosphonium chloride (THPC, 80% in water) were purchased from Sigma-Aldrich. Formaldehyde solution (CH₂O, 31%) was purchased from Macron Fine Chemicals. Methoxy PEG Thiol (mPEG-SH) (MW=2,000) was purchased from Laysan Bio, Inc.

1-wt % chloroauric acid solution was prepared by suspending 5 grams of gold (III) chloride trihydrate in 500 mL Milli-Q water. The chloroauric acid solution was aged for at least one month before use.

Multi-element internal standard (2-wt % HNO₃, 10 mg/L Ho) were purchased from Atomic Spectroscopy. Gadolinium ICP/DCP standard solution (10,006 μg/mL in 2-wt % HNO₃) and hydrochloric acid (HCl, ≥30%) were purchased from Fluka Analytical. Sodium hydroxide solution (NaOH, 1 N), potassium carbonate anhydrous (K₂CO₃, ≥99%), and nitric acid (HNO₃, 70%) were purchased from Fisher chemical.

Aqua regia (HNO₃/HCl (v/v), 1:3) was used to clean laboratory glassware and stir bars, followed by thorough rinsing with DI-Water. Milli-Q water (18.2 MΩ·cm at 25° C., Millipore) was used during all of the reactions and the last step of glassware washing.

### Synthesis of Inner Layer Comprising a Dielectric Core

Silica core was synthesized by a modified Stöber process. 0.383 g of cetyltrimethylammonium chloride which may act as structure-directing agent, was mixed with 175 mL Milli-Q water at 30-35° C. in a 500 mL closed round beaker connected to a condenser to avoid solution depletion until the CTAC was completely dissolved. Under continuous stirring, 75 mL of 200 proof ethanol was added for a 250 mL final synthesis volume followed by the addition of 300 μL of ammonia as the catalyst to reach a pH of 10, followed by 200 μL of tetraethyl orthosilicate as silica source.

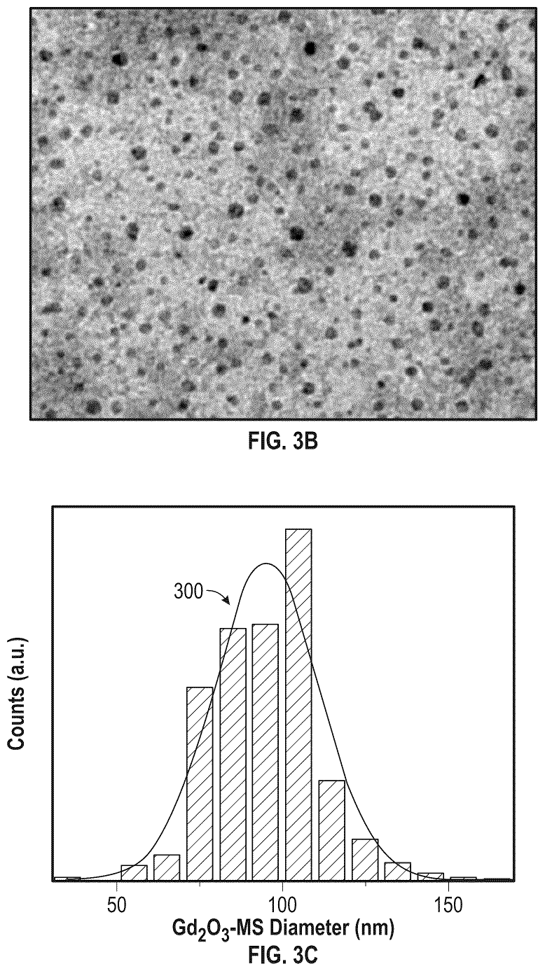

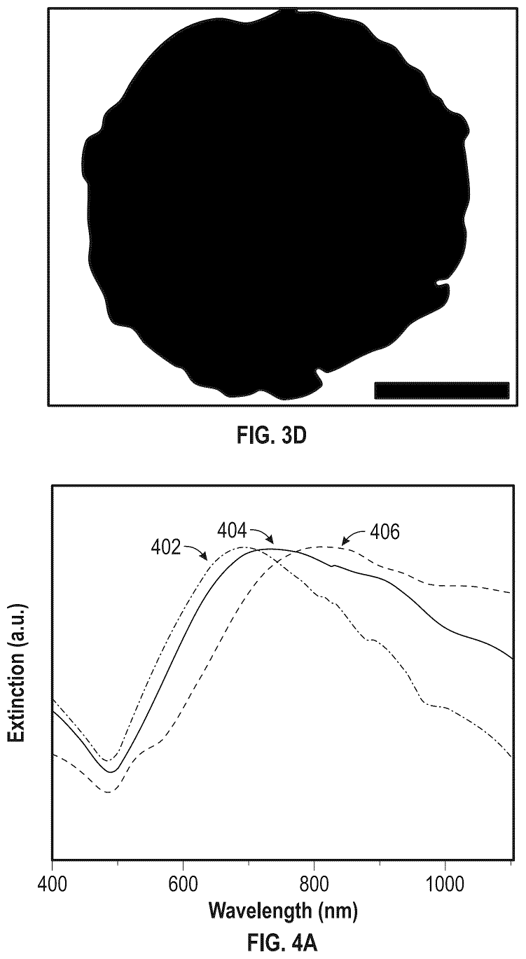

The TEOS solution was added dropwise under continuous stirring to grow particles with a narrow size distribution. The reaction was run at 60° C. for three days. The reaction was cooled down to room temperature while continuously stirring to avoid particle aggregation. The particles were recovered by centrifuging a 10 mL sample in a 50 mL centrifuge tube for 15 minutes at 18,000 g and 25° C. The collected pellet was dispersed in ethanol with mild sonication followed by three centrifugation cycles to remove free TEOS. The final pellet was dispersed in approximately 1 mL of water. The size distribution of the silica nanoparticles is determined by transmission electron microscopy. FIG. 3A is an exemplary TEM image of a plurality of silica nanoparticles synthesized.

### Synthesis of Porous Substrate

The dispersed final pellet with the silica core nanoparticles was baked at 500° C. for 4 hours to remove the CTAC template. The remaining final white solid material of mesoporous silica was dispersed in 5 mL of Milli-Q water through vigorous sonication to generate mesoporous silica (MS) as the porous substrate.

Nitrogen adsorption-desorption isotherm measurements and Brunauer-Emmett-Teller (BET) data indicate that the MS cores have a surface area of 907 m²/g with an average diameter pore size of 2.5±0.5 nm. Adsorption is defined as the adhesion of atoms or molecules of gas to a surface. The amount of gas adsorbed depends on the exposed surface area. During BET analysis, the amount of gas adsorbed on a surface is measured.

### Synthesis of Magnetically Responsive Nanoparticles

According to one or more embodiments, ultrasmall Gd₂O₃ nanoparticle (NP) synthesis was performed under an argon environment attached to the condensation system to better control the oxidation process. 5.8 g of gadolinium (III) chloride hexahydrate was dissolved in 100 mL of diethylene glycol in a 250 mL round beaker. The solution was heated to at 60° C., the temperature was maintained, and the solution was stirred overnight. 22.5 mL of 1 N NaOH was added quickly and vigorously stirred at 750 rpm while the temperature was increased to 140° C. at 5° C./min ramping rate (RR). After 1 hour of reaction at 140° C., the temperature was increased to 180° C. (RR=4° C./min) and the reaction was continued for 4 hours. The final product was a transparent colloid solution of Gd₂O₃ nanoparticles (NPs). The suspension of Gd₂O₃ was cooled down to room temperature and stored at 4° C.

The average hydrodynamic diameter of the synthesized Gd₂O₃ nanoparticles was 3.0±0.3 nm, as determined by dynamic light scattering measurements and TEM images. FIG. 3B is an exemplary TEM image of a plurality of synthesized Gd₂O₃ nanoparticles.

Loading Magnetically Responsive Nanoparticle into Porous Dielectric Substrate

5 mL of the mesoporous silica (MS) solution from above and 5 mL of Gd₂O₃ NPs solution from above were mixed and sonicated for 4 hours to facilitate penetration of Gd₂O₃ into the mesoporous silica pores. The suspension was settled overnight, and the supernatant was collected. In a 50 mL centrifuge tube, 5 mL of supernatant was centrifuged at 18,000 g for 15 min. The pellet was collected and redispersed in water for two rounds of centrifugation to remove free Gd₂O₃ NPs. The Gd₂O₃-mesoporous silica nanoparticle pellet was then redispersed in 40 mL of ethanol in a polypropylene flask and stirred rapidly for 10 minutes.

### Linker Attachment to Magnetically Responsive Nanoparticle Loaded Porous Dielectric Substrate

After stirring for 10 minutes, 200 μL of APTES was added to the redispersed pellet. This step may create aminated sites on the Gd₂O₃-MS surface that may facilitate the attachment of gold NPs in the subsequent synthesis step. The reaction was continued overnight under slow stirring speed at room temperature. Next, the mixture was transferred to a borosilicate glad container and boiled at 85° C. for one hour with the addition of ethanol to prevent solution depletion. The aminated Gd₂O₃-MS suspension was centrifuged three times at 18,000 g for 15 minutes and redispersed in ethanol. The final amine-terminated Gd₂O₃-MS was dispersed in ethanol. FIG. 3C is an exemplary histogram plot showing the average size distribution of aminated Gd₂O₃-MS NPs. The total number of Gd₂O₃-MS NPs measured to generate the plot were 2,930. The average diameter of the Gd₂O₃-MS NPs was determined to be 95±nm by a Gaussian distribution fit (300).

### Metal Nanoparticle Synthesis

Under rapid stirring, 1.2 mL of 1 M NaOH is added to 180 mL of Milli-Q water, followed by 4 mL of 1.2% volume aqueous solution of tetrakis(hydroxymethyl) phosphonium chloride (THPC, 80% in water, Sigma Aldrich) in 33 mL of DI-water. The reaction was run for 5 minutes. Next, 6.75 mL of 1% wt of 1-month aged chloroauric acid (prepared by suspending 5 g of gold (III) chloride trihydrate, ≥99.9%, Sigma-Aldrich in 500 mL Milli-Q water) solution was added, turning the solution from clear to brown color. After 10 minutes of vigorous stirring, the final suspension was stored at 4° C. for a specified amount of time before being used to form the metal shell.

### Metal Nanoparticles Attachment to Magnetically Responsive Nanoparticle Loaded Porous Dielectric Substrate

40 mL of 1-3 nm gold NPs (one week aged) were sonicated for 5 minutes. During sonication, 600 μL of 1M sodium chloride and 1 mL of aminated Gd₂O₃-MS solution were added to the mixture, vortexed, sonicated for 30 minutes, and stirred overnight at room temperature to form a Au-seed Gd₂O₃-MSs solution. The 40 mL Au-seed Gd₂O₃-MSs solution was centrifuged at 1,000×g for 30 min. The pellet was collected and resuspended in 10 mL water followed by mild sonication and then centrifuged at 400×g for 30 minutes. Another round of resuspension in water, sonication, and centrifugation was repeated to remove free-gold NPs. The final pellet was suspended in 5 mL of DI water. The 1-3 nm Au NPs attached to the Gd₂O₃-MSs surface may act as nucleation sites for the electroless deposition of gold to form a complete shell on the porous silica core.

### Metal Shell Deposition on Magnetically Responsive Nanoparticle Loaded Porous Dielectric Substrate

In a dark glass container, a plating solution was prepared by adding 50 mg of potassium carbonate to 200 mL of Milli-Q water followed by 3 mL of 1% wt chloroauric gold solution. The plating solution was shaken for approximately 1 minute and left in the dark overnight. The gold-seed Gd₂O₃-MSs solution from was refreshed by 15 min of sonication. The gold shell was grown around the Gd₂O₃-MSs cores via seed-mediated electroless plating. The electroless deposition was achieved by reducing gold from a 1.8 mM potassium carbonate solution and 0.4 μM chloroauric acid by formaldehyde. 3 mL of plating solution was added to 22 μL of Au-seeds Gd₂O₃-MS aqueous suspension followed by the addition of 15 μL 31% formaldehyde solution. The solution was shaken and sonicated until a continuous gold shell is formed around the Au-seeded Gd₂O₃-MS NPs, causing the solution color to change from light red to light blue and the absorption resonance to shift from the UV range to the NIR (700 nm-800 nm), respectively.

By varying the volume of Au-seeds Gd₂O₃-MS NPs, the gold shell thickness was tuned to match the extinction spectrum for a maximum at the 810 nm laser wavelength. After the right volume of Au-seeded Gd₂O₃-MS was determined, the reaction was scaled up by running multiple reactions. Every five cuvettes (3 mL each) were combined in a 50 mL centrifuge tube and centrifuged at 350×g for 30 minutes while avoiding composite nanoparticle aggregation. The pellets were collected and redispersed in water, mildly sonicated, recentrifuged for two more times.

To prevent particle aggregation, the composite nanoparticles were redispersed in 25 mL of Milli-Q water in a 50 mL centrifuge tube and mixed with 0.5 mL of 1 mM mPEG-SH solution (2 kDa). The mixture was sonicated for 20 minutes and left on a shaker overnight horizontally at a ˜20° degree angle. Next, the suspension was sonicated for 20 minutes and was settled down on a bench for 30 min. Then, it was collected and is centrifuged at 350×g for 30 minutes. The pellets were collected, redispersed in Milli-Q water, mildly sonicated, and centrifuged for two rounds to remove free PEG and gold seeds. The pellet was resuspended in 10 mL Milli-Q water and stored in the refrigerator for months.

The photothermal magnetic resonance imaging contrast enhancement agent is mono-dispersed in size and the outer dimension of each composite nanoparticle is less than 150 nm. FIG. 3D is an exemplary TEM image of a composite nanoparticle synthesized with a scale bar of 50 nm.

### Extinction as a Function of Metal Shell Thickness

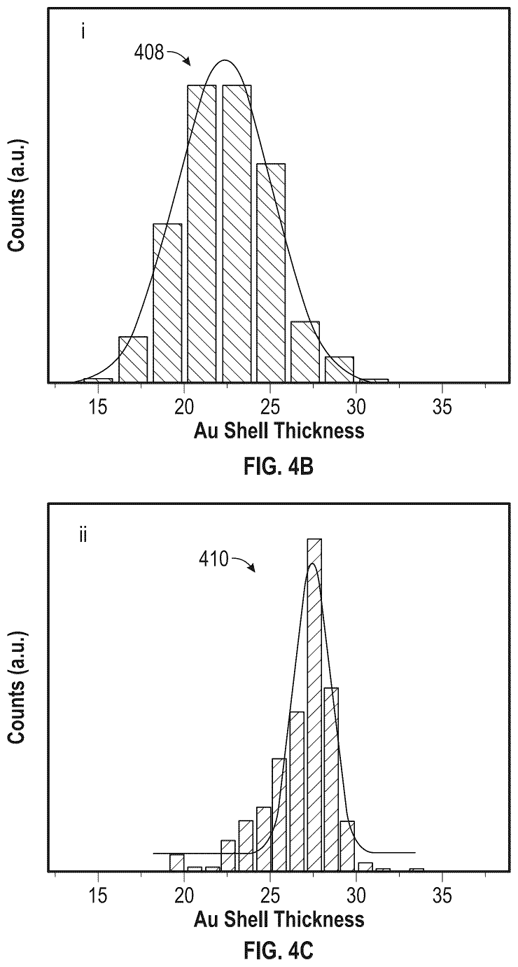

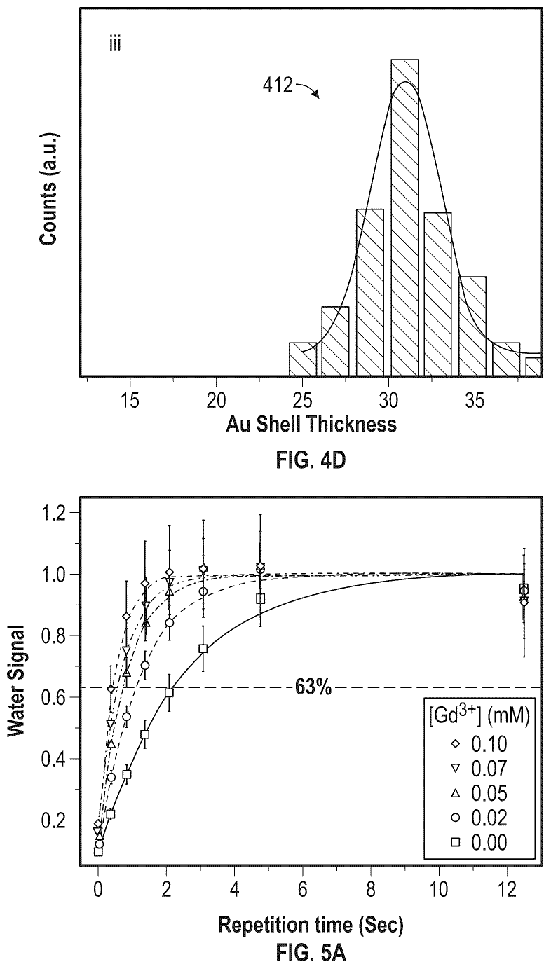

According to one or more embodiments, the plasmon resonance of the photothermal magnetic resonance imaging contrast enhancement agent may be tuned from 600 nm to about 900 nm by varying the metal shell thickness when measured. The extinction spectra of three aqueous suspension of PEGylated composite nanoparticles with varying metal shell thicknesses are shown in FIG. 4A. The extinction spectra reveal a blue shift in the plasmon resonance with an increase in gold shell thickness, as predicted by Mie theory. FIG. 4A shows the extinction spectra of composite nanoparticles with an average metal shell thickness of 22 nm (402), 27.5 nm (404), and 31 nm (406).

The average metal shell thickness was determined by high resolution transmission electron microscopy. The plot in FIG. 4B was obtained by imaging and measuring the diameter of 304 composite nanoparticles. FIG. 4B corresponds to a shell thickness of 22 nm and spectrum 402 in FIG. 4A. The plot in FIG. 4C was obtained by imaging and measuring the diameter of 339 composite nanoparticles. FIG. 4C corresponds to a shell thickness of 27.5 nm and spectrum 404 in FIG. 4A. The plot in FIG. 4C was obtained by imaging and measuring the diameter of 297 composite nanoparticles. FIG. 4C corresponds to a shell thickness of 31 nm and spectrum 406 in FIG. 4A. The composite nanoparticle distributions were fit using a Gaussian function to obtain the shell thicknesses. In FIG. 4B the Gaussian fit (408) determined the average shell thickness to be 22±2.5 nm. In FIG. 4C the Gaussian fit (410) determined the average shell thickness to be 27.5±0.5 nm. In FIG. 4D the Gaussian fit (412) determined the average shell thickness to be 31±2 nm.

### Gadolinium Ion Concentration

The MRI contrast was measured as a function of the Gd³⁺ concentration. The concentration of Gd³⁺ within composite nanoparticle samples was measured using a Perkin Elmer Nexion 300 ICP-MS. Initially, 25 μL of each composite nanoparticle sample was digested in 200 μL concentrated aqua regia and left overnight with a loss cover. The resulting solutions were diluted with 2% v/v nitric acid (HNO₃) by 400 times. Furthermore, various Gadolinium ICP/DCP standard solution concentrations with 1, 10, 100, and 1,000 μg/L were prepared to generate a calibration curve. Internal standard (Ho 165) was added to all samples, standard solutions, and a blank solution and kept its final concentration the same (15 μg/L) to ensure no changes within the instrument detection sensitivity occurred during the measurements.

### MRI Property of Composite Nanoparticles

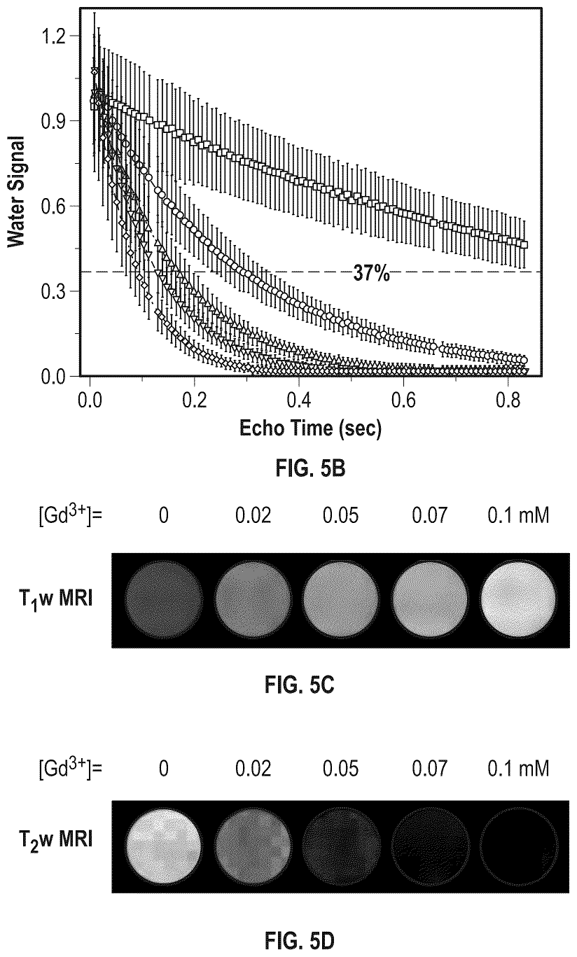

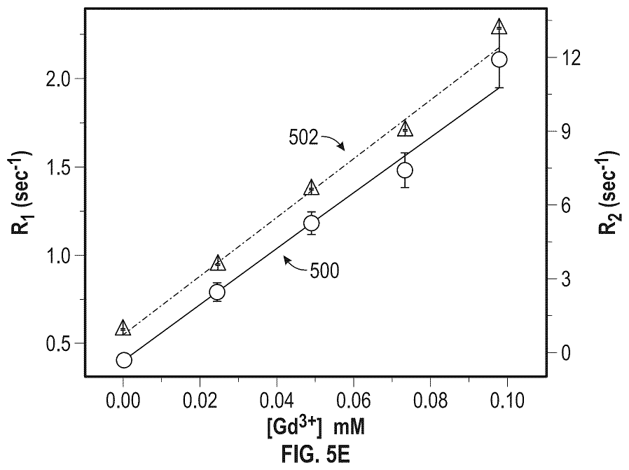

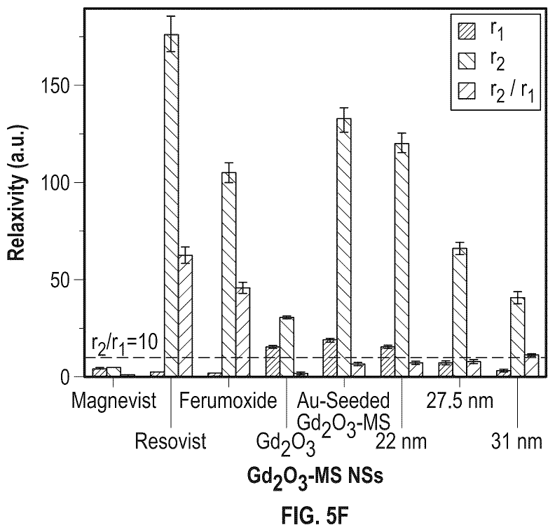

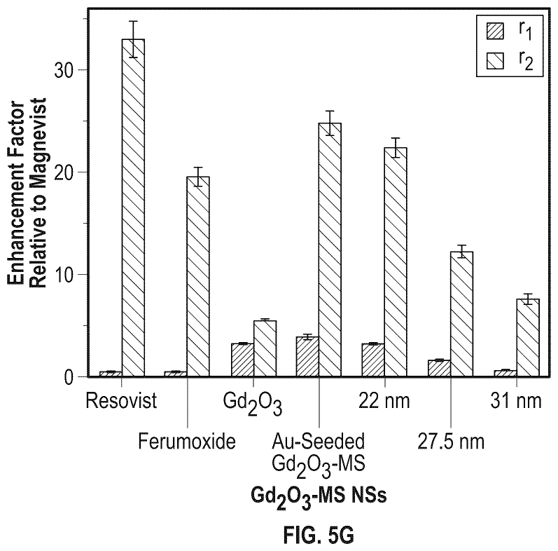

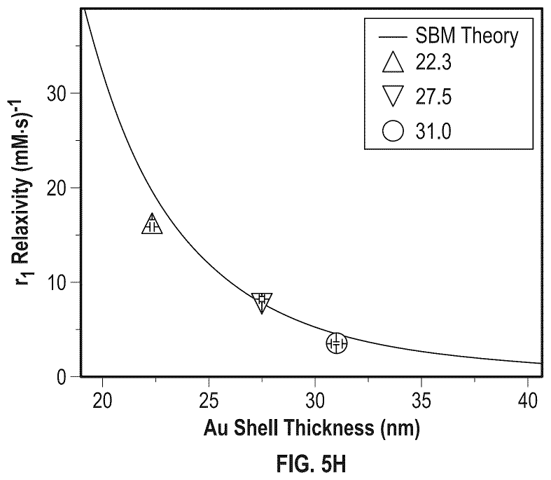

The MRI T₁ and T₂ contrast of composite nanoparticles with a gold shell thickness of 22 nm was determined at 4.7 Tesla by measuring the recovery of longitudinal magnetization (Mz) at various repetition times (TR) and the decay of transverse magnetization (Mxy) at multiple echo times (TE) of water protons in the presence of numerous Gd³⁺ concentrations as shown in FIG. 5A and FIG. 5B. The plots in FIG. 5A and FIG. 5B show that increasing the Gd³⁺ concentration (corresponding to curves from top to bottom in FIG. 5B, respectively) shortens both T₁ and T₂ relaxation times of the neighboring water protons.

Equations 1-2 were used to determine the longitudinal (T₁) and transverse (T₂) relaxation time constants, respectively:

$${M}_{z} = {M}_{{z}_{0}} ( 1 - {e}^{- TR / {T}_{1}} ) \qquad ( 1 )$$

$${M}_{xy} = {M}_{{xy}_{0}} {e}^{- TE / {T}_{2}} \qquad ( 2 )$$

Where M_{z}_{₀} and M_{xy}_{₀} are scaling factors.

Increasing Gd³⁺ concentration shortens both T₁ and T₂ relaxation times of the neighboring water protons, causing an increase in signal intensity at the T_{1w} MRI and a decrease in signal intensity at the T_{2w} MRI respectively. FIG. 5C and FIG. 5D show the change in T_{1w} and T_{2w} MRI signal intensity of 22 nm shell thick composite nanoparticles respectively. The T_{1w} MRI and the T_{2w} MRI were obtained with RAREVTR scanning (TR=400 ms, TE=9.9 ms, FA=) 180° and MSME scanning (TR=1,750 ms, TE=423 ms, FA=) 180°, respectively.

FIG. 5E shows that plotting R₁ (1/T₁) (500) and R₂ (1/T₂) (502) relaxation rate constants as a function of Gd³⁺ concentrations for composite nanoparticles with a 22 nm gold shell showed a linear dependence with relaxivity rates (slope) values of r₁=17±1 mM_{Gd}⁻¹·s⁻¹ and r₂=122±7 mM_{Gd}⁻¹·s⁻¹. These measurements showed that the r₂/r₁ ratio is 7.2, which may be ideal for a T₁ contrast agent. Generally, a r₂/r₁ ratio of <10 is ideal for T₁ contrast agents.

FIG. 5F is an exemplary plot of r₁ relaxivity rate (left bar), r₂ relaxivity rate (middle bar), r₂/r₁ ratio (right bar) of Magnevist®, Resovist®, Ferumoxide, Gd₂O₃ nanoparticles, Gd₂O₃-MS with Au seed, and composite nanoparticles with three different shell thicknesses (22 nm, 27.5 nm, and 31 nm).

First, the measured r₁ (16±5 mM_{Gd}⁻¹·s⁻¹) and r₂ (30±5 mM_{Gd}⁻¹·s⁻¹) relaxivity values of the synthesized Gd₂O₃ at 4.7 T was comparable to literature values of r_{1=8.8} mM⁻¹·s⁻¹ (approximately 2.2 nm diameter, 7 T) and r_{1=14.9} mM_{Gd}⁻¹·s⁻¹ (approximately 2 nm diameter, 0.5 T). FIG. 5F shows that loading synthesized Gd₂O₃ nanoparticles onto the mesoporous silicon core to form composite nanoparticles with a gold shell thickness of 22 nm did not affect the r₁, but enhanced r₂ by a factor of four.

The r₁ relaxivity value, r₂ relaxivity value, and r₂/r₁ ratio at various stages of the composite nanoparticle synthesis is shown in FIG. 5F. FIG. 5F further shows the r₁ relaxivity value, r₂ relaxivity value, and r₂/r₁ ratio of composite nanoparticles with three different shell thicknesses, Magnevist®, Ferumoxide, and Resovist®, Gd₂O₃ nanoparticles, and Gd₂O₃-MS with Au seed.

The r₁ and r₂ relaxivity rate at various stages of the composite nanoparticle synthesis is shown in Table 1 below.

FIG. 5F and Table 1 show that Gd₂O₃ nanoparticles, Gd₂O₃ nanoparticles containing nanoparticles, and composite nanoparticles have a higher r₁ relaxivity rate compared to a standard T₁ MRI contrast agent like Magnevist®. Table 1 shows that the r₁ of composite nanoparticles with 22 nm shell is 3.6×r₁ relaxivity rate of Magnevist® (T₁ MRI contrast agent).

Notably, FIG. 5F and Table 1 also show that composite nanoparticles with shell thickness of 22 nm and 27.5 nm have comparable r₂ relaxivity rate compared to standard T₂ MRI contrast agents based on superparamagnetic iron oxide (SPIO) such as Ferumoxide (105 mM_{Fe}⁻¹·s⁻¹) and Resovist® (176 mM_{Fe}⁻¹·s⁻¹).

TABLE 1

| Sample | Slope, r₁ (mM⁻¹ · s⁻¹ ) | R² | Slope, r₂ (mM⁻¹ · s⁻¹ ) | R² |
|---|---|---|---|---|
| Magnevist ® | 4.7 ± 0.1 | 0.996 | 5.34 ± 0.04 | 0.999 |
| Gd₂O₃ nanoparticles | 15.8 ± 0.7 | 0.992 | 30.6 ± 0.3 | 0.999 |
| Gd₂O₃-MS with Au seed | 19 ± 1 | 0.98 | 132 ± 6 | 0.99 |
| Composite nanoparticles | 17 ± 1 | 0.98 | 122 ± 7 | 0.99 |
| with 22 nm shell | | | | |
| thickness | | | | |
| Composite nanoparticles | 8.5 ± 0.4 | 0.993 | 68 ± 13 | 0.993 |
| with 27.5 nm shell | | | | |
| thickness | | | | |
| Composite nanoparticles | 4.6 ± 0.1 | 0.996 | 42 ± 3 | 0.996 |
| with 31 nm shell | | | | |
| thickness | | | | |

FIG. 5G is a plot of the enhancement factor relative to Magnevist®.

When multiple domains of paramagnetic material such as Gd₂O₃ are concentrated within a single structure, as in composite nanoparticles, it is possible that the entire composite nanoparticles NP structure starts to behave as a superparamagnetic material. Multiple domains of Gd₂O₃ in a single composite nanoparticle may increase the susceptibility of the composite nanoparticle, making them more magnetic when placed in an external magnetic field, improving the composite nanoparticle interaction with the external magnetic field, causing the magnetic lines to be even more concentrated within the composite nanoparticles. This increases the local magnetic field differences and the composite nanoparticles to have superparamagnetic properties.

Unlike T₁ relaxation time, T₂ relaxation time is sensitive to local changes in the magnetic field. The more perturbation within the local magnetic field, each spin will experience different local magnetic fields, causing them to process at various frequencies, increasing decoherence and inhomogeneity in the transverse plane, and eventually a faster decay of the transverse magnetization is detected. This could explain the 4.4 fold of enhancement in the r₂ of composite nanoparticles with 22 nm gold shell, for instance, relative to Gd₂O₃. The composite nanoparticles with 22 nm and 27.5 nm gold shells were used for further experimentation since both provided sufficient contrast enhancement and a strong NIR plasmon resonance sufficient to perform MRI-guided PTT.

The measure values of r₁ relaxivity values of Gd₂O₃-MS NPs were compared to theoretical calculations, as shown in FIG. 5H. The best agreement between the theoretical calculations and the measured values of r₁ relaxivity values of Gd₂O₃-MS was achieved when 50% of the Gd₂O₃ NPs were distributed on the mesoporous silica (MS) surface and the other 50% were distributed uniformly inside the MS core. Therefore, the measurements are consistent with an arrangement of magnetically responsive nanoparticles in which 50% of the magnetically responsive nanoparticles may be distributed in an outer portion of the inner layer and 50% may be uniformly distributed in the bulk of the inner layer.

Ratio of T_{1w}/T_{2w} Signal Intensities with Composite Nanoparticles

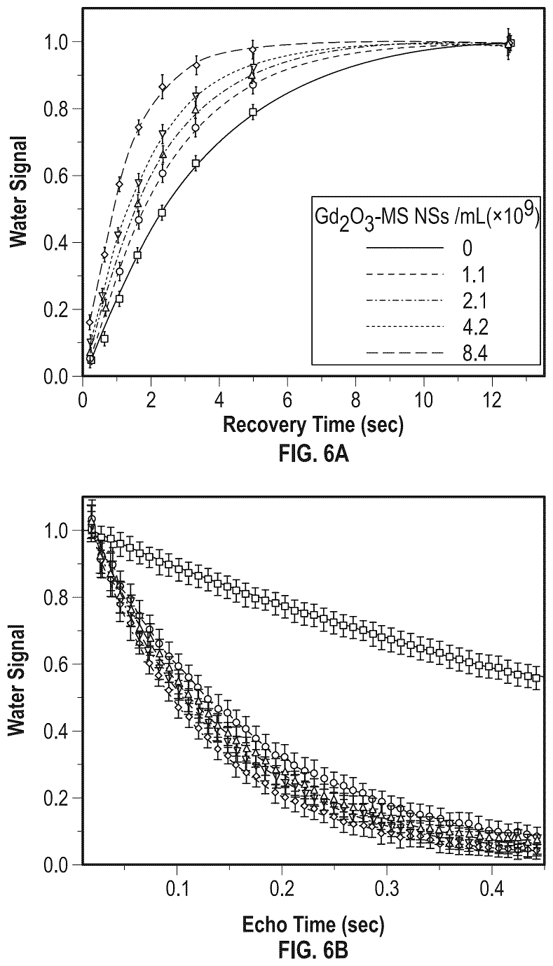

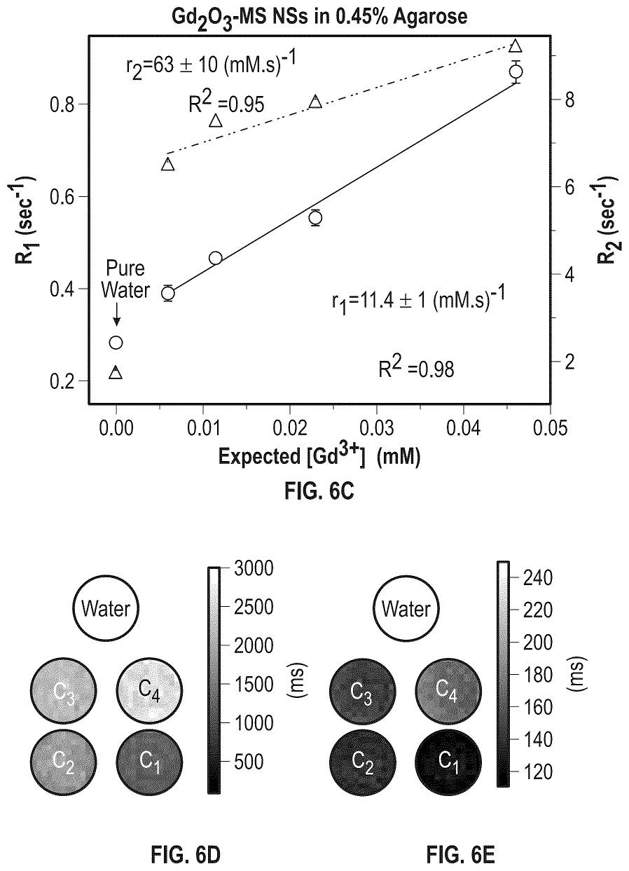

FIGS. 6A-E shows the MRI measurements for composite nanoparticles with 27.5 nm shell at four different concentrations in 0.48% agarose phantoms used for thermal MRI mapping study performed at 4.7 T.

Four NMR tubes were prepared with a 40 μL layer of 1.1×10⁹ (C₄), 2.1×10⁹ (C₃), 4.2×10⁹ (C₂), and 8.4×10⁹ (C₁) composite nanoparticles/mL with 27.5 nm shell thickness in 0.48% agarose surrounded with 0.8% agarose suspension. The NMR tubes were placed in a square shape with an empty tube in the middle to position and center the diffuser optical fiber with an equal distance from all samples and the same laser power density exposure. A control sample containing water and without composite nanoparticle was used. FIGS. 6A and 6B show plots of longitudinal recovery and transverse decay, respectively, for water and C₁-C₄ samples with increasing concentration (corresponding to curves from top to bottom in FIG. 6B, respectively).

Their T₁ (FIG. 6D) and T₂ (FIG. 6E) maps were established and their MRI properties were evaluated, giving a stable and reproducible r₁ (11.8±0.9 mM_{Gd}⁻¹·s⁻¹) and r₂ (61±11 mM_{Gd}⁻¹·s⁻¹) relaxivity rates at ten months post-synthesis.

As observed earlier, increasing composite nanoparticles improved the signal contrast (SC) in T_{1w} and T_{2w} images by enhancing the signal in T_{1w} MRI and suppressing that in T_{2w} MRI. By taking the ratio of T_{1w}/T_{2w} signal intensities, the obtained processed MRI gave a better contrast with a higher signal-to-noise ratio. For processing the MR image intensity with the T_{1w}/T_{2w} ratio, the Tw and T_{2w} images with the optimum SC were applied.

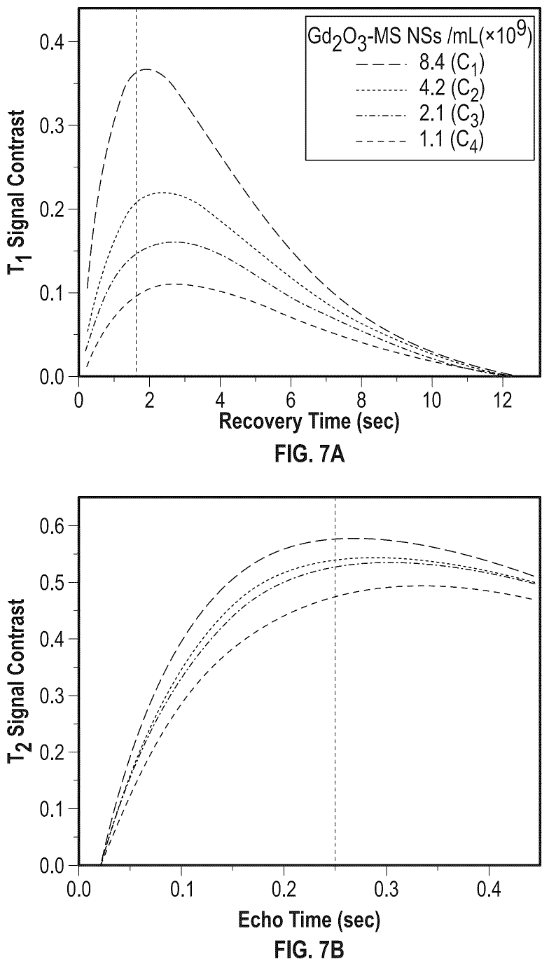

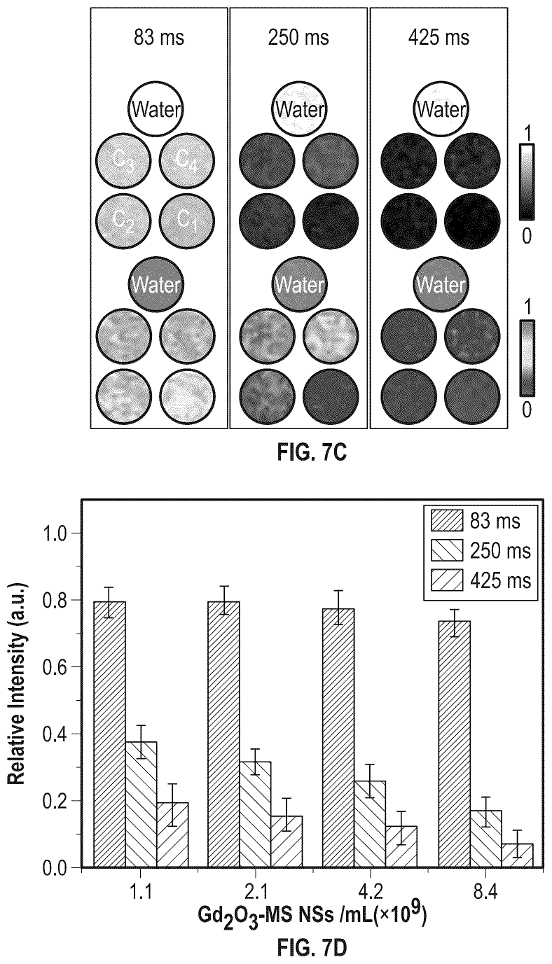

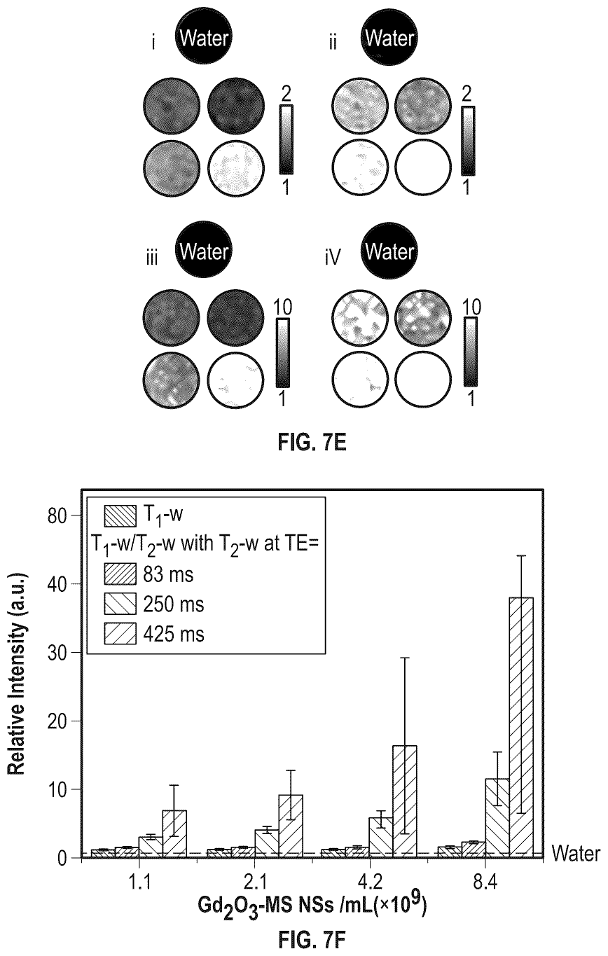

The optimum T₁ and T₂ SC were established using T_{1w} MRI with TE=9.9 ms and TR=1,580 ms, and in T_{2w} MRI with TE=250 ms and TR=3,857 ms, respectively (FIGS. 7A-B). This was determined by subtracting the fitted signals for each of the composite nanoparticles dilutions from that for the reference (water) over a range of TE and TR values (FIGS. 6A-E). The T_{2w} image with a shorter echo time (e.g. TE=83 ms) was also considered because it is more likely to be used in the clinic. Increasing the TE from 83 ms to 250 ms and 450 ms increased the contrast and suppressed the signal intensity in T_{2w} MRI with increasing composite nanoparticles NSs concentrations (FIGS. 7C-D). With 1.1×10⁹ composite nanoparticles/mL (0.006 mM estimated Gd³⁺), the signal intensity of water in T_{1w} MRI was increased by 1.26 fold (FIG. 7F) while in T_{2w} MRI was reduced to 80%, 38%, and 19% at TE=80 ms, 250 ms, and 450 ms, respectively (FIG. 6D). Nevertheless, processing the T_{1w}/T_{2w} intensity ratio enhanced the contrast to water by 1.6±0.1, 3.4±0.5, and 8±4 fold (mean±Std.Dev.) with T_{2w} MR images at TR=3,857 ms and TE=80 ms, 250 ms, and 450 ms, respectively (FIGS. 7E-F). High standard deviation values for the processed T_{1w}/T_{2w} ratio were evident due to T_{2w} images increased sensitivity to noise at longer echo times.

### Photothermal Therapy Using Composite Nanoparticles

In MRI, the shift in the proton resonance frequency of water is temperature-sensitive. Slight changes in the resonance frequency caused by changes in temperature may lead to a phase change in the MR images; the temperature change at an image N from the baseline (T_{N}−T₁) is a function of the phase change (80) based on the relationship in Equation 3.

$${T}_{N} = {T}_{1} = {\sum }_{i = 1}^{N} {T}_{i} - {T}_{i - 1} = \frac{{\sum }_{i = 1}^{N} {δϕ}_{i}}{2 π \cdot γ {B}_{o} \cdot α \cdot TE} \qquad ( 3 )$$

Where α is an assumed temperature sensitivity (−0.01 ppm/° C.), and TE is the sequence echo time (6 ms). The proton resonance frequency (ω₁) is the product of y the gyromagnetic ratio (42.58 MHz/T for hydrogen) and B₀ magnetic field strength (4.7 T).

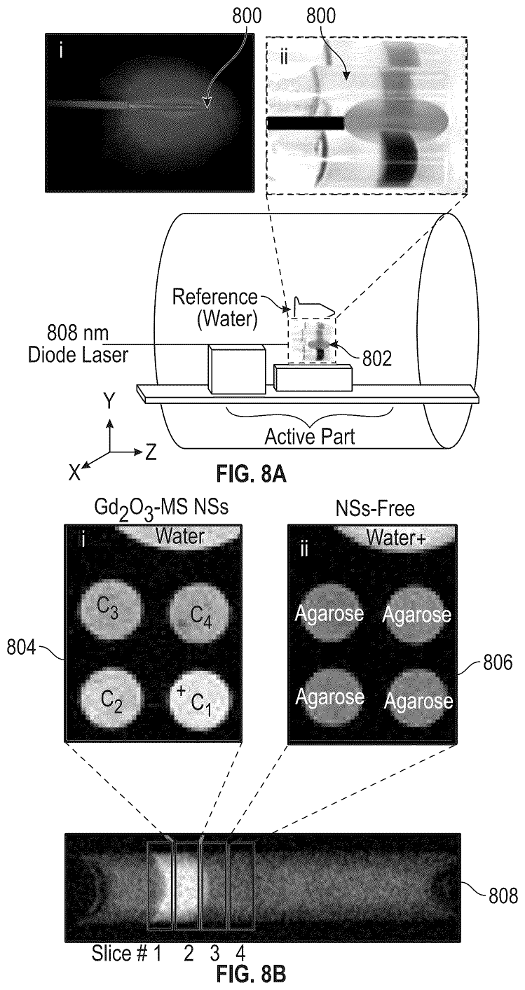

A 10 mm diffuser tip was used to deliver an 808 nm illumination source 802 (CW-GaA1As Laser diode) with a uniform power density across the NMR tubes while monitoring the temperature change with thermal MRI mapping performed at 4.7 T (FIG. 8A). Before laser illumination, T_{1w} and T_{2w} MR images were acquired to localize the treatment and establish thermal-image planes. FIG. 8B is a coronal T_{1w} MRI for one of the NMR tubes with the relevant slices set for thermal MRI measurements to monitor the temperature change during laser illumination. The superscript images were axial T_{1w} MR images of slice #2 and #4 across the NMR tubes at composite nanoparticle 804 locations and composite nanoparticle free 806 agarose medium, respectively. A series of 14 T_{1w} MR images were acquired (73 sec/image) to monitor and control the laser treatment in real-time. This set of images covered a baseline, 3 min of 808 nm illumination at 4 W, and temperature recovery measurements. The T_{1w} MR images were processed with a MATLAB code to generate a thermal map for any plane of interest.

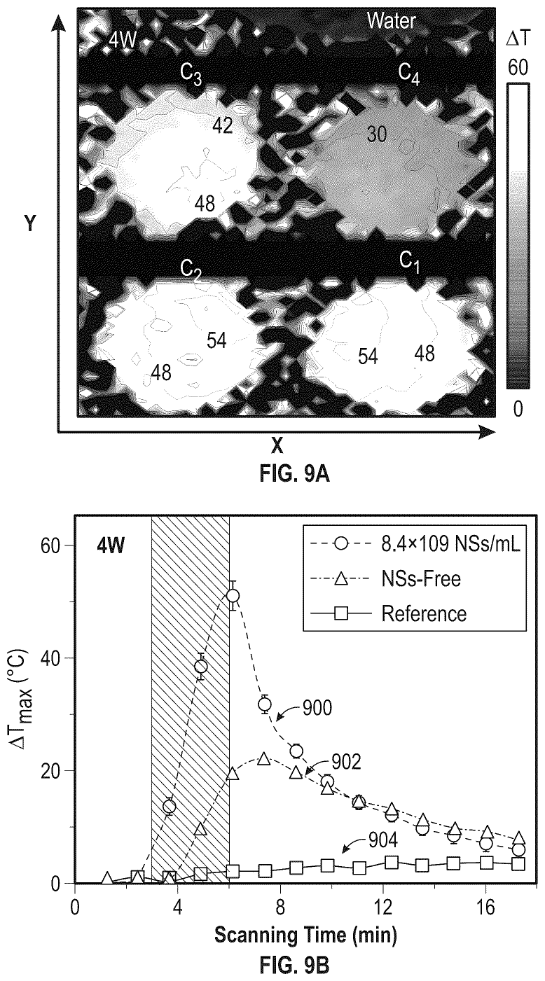

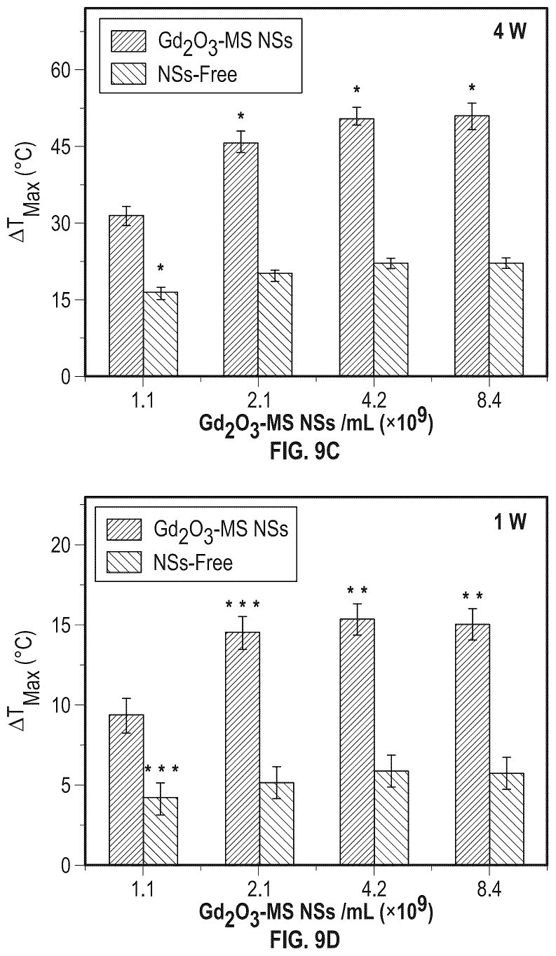

FIG. 9A is a thermal map obtained from the last FLASH image acquired before the laser was turned OFF, presenting the maximum temperature change at the composite nanoparticle location. After three minutes of continuous NIR-illumination at 4 W, the highest temperature change (30° C.≤ΔTₘₐₓ≤54° C.) at the composite nanoparticle locations was achieved close to the illumination source.

FIG. 9B is the temperature change profile in real-time during MR thermometry at three regions of interest 1) composite nanoparticle with 8.4×10⁹ composite nanoparticle/mL (900), 2) approximately 2 mm below the composite nanoparticle band in the same NMR tube (902), and 3) reference (904), reaching a maximum temperature change of 54° C., 20° C., and ˜4° C., respectively. A rapid increase in the temperature (18° C./min) was detected at the composite nanoparticle locations as soon as the laser illumination was ON (FIG. 9B).

Increasing composite nanoparticle concentrations increased the localized generated heat until a plateau is reached as shown in FIGS. 9C-D. This observation was consistent with previous reports within the same range of concentrations (≥4×109 nanoparticles/mL). A maximum temperature change of 54° C. was reached for composite nanoparticles with 4.2×10⁹ and 8.4×10⁹ composite nanoparticle/mL concentrations, while 30° C. was achieved for the lowest concentration (1.1×10⁹ composite nanoparticle/mL). Another slice of NS-free agarose medium was used to monitor the temperature, ˜2 mm lower than the composite nanoparticles band, revealing a maximum temperature change of ˜ 20° C., weakly increasing with temperature change and composite nanoparticle concentrations in the nearby layer (FIG. 9C, dashed columns). As soon as the illumination source was turned OFF, it took about four minutes for the temperature to go down and equilibrate with the surrounding composite nanoparticle-free agarose medium (FIG. 9B). The same experimental setup was performed at lower laser power (1 W). A maximum temperature increase of 10-17° C. was caused by (1.1-8.4)×10⁹ composite nanoparticles/mL while inducing a maximum temperature change of 5° C. to the nearby composite nanoparticle-free medium (˜ 2 mm down, FIG. 9D).

Three minutes with 808 nm at 4 W with 10 mm diffuser tip, may cause excessive thermal damage, resulting in a temperature increase of between about 20° C. to about 54° C. at the composite nanoparticle location with 1.1-8.4×10⁹ composite nanoparticle/mL and temperature increase of about 15° C. to 20° C. for the nearby nanoparticle-free agarose phantom. In vivo, a temperature increase of about 12° C. reaching a body temperature of greater than 49° C. may be sufficient to induce thermal damage by necrosis even in NS-free tissue regions, as it may destroy the cell membrane, secrete the cytoplasm to the extracellular part, and induce inflammation. Thus, reducing laser power to levels below what has been used in clinical studies is critical for minimal thermal heating of the NS-free agarose medium. Decreasing the laser power to 1 W is sufficient to cause thermal heating at the composite nanoparticle locations (9-17° C.) in the agarose phantom while causing minimum thermal heating to the NS-free agarose phantom (ΔTₘₐₓ=5° C.). These temperature changes may initiate apoptosis and necroptosis, ideal for tumor thermal ablation while sparing healthy tissue.

To be used in vivo applications, the photothermal Magnetic Resonance Imaging (MRI) enhancement agent may be suspended in an appropriate solution to form an injectable formulation.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A photothermal magnetic resonance imaging enhancement agent, comprising:
a plurality of composite nanoparticles, each composite nanoparticle comprising:
an inner layer comprising a dielectric material comprising a porous substrate having pores;
a plurality of magnetically responsive nanoparticles disposed on the porous substrate; and
an outer layer comprising a metallic material around the inner layer and the magnetically responsive nanoparticles.

2. The photothermal magnetic resonance imaging enhancement agent of claim 1, wherein an average outer diameter of the inner layer is between about 80 nm and about 110 nm.

3. The photothermal magnetic resonance imaging enhancement agent of claim 1, wherein the inner layer comprises a dielectric core.

4. The photothermal magnetic resonance imaging enhancement agent of claim 1, wherein the inner layer comprises a dielectric layer disposed around a metallic core.

5. The photothermal magnetic resonance imaging enhancement agent of claim 1, wherein the magnetically responsive nanoparticles have an average diameter between about 2 nm and about 3 nm.

6. The photothermal magnetic resonance imaging enhancement agent of claim 1, wherein the magnetically responsive nanoparticles comprise gadolinium oxide.

7. The photothermal magnetic resonance imaging enhancement agent of claim 1, wherein the composite nanoparticle is selected from the group consisting of a type 1 (T₁) contrast agent and a type 2 (T₂) contrast agent.

8. The photothermal magnetic resonance imaging enhancement agent of claim 5, wherein the composite nanoparticle has a relaxivity rate r₁ of at least 3.6 times greater than a reference gadopentetate dimeglumine T₁ MRI contrast agent.

9. The photothermal magnetic resonance imaging enhancement agent of claim 8, wherein the composite nanoparticle has a relaxivity rate r₂ comparable to a reference superparamagnetic iron oxide T₂ MRI contrast agent.

10. The photothermal magnetic resonance imaging enhancement agent of claim 8, wherein the composite nanoparticle is a type 1 contrast agent and a type 2 contrast agent.

11. The photothermal magnetic resonance imaging enhancement agent of claim 1, wherein a surface area of the porous substrate is between about 900 m²/g to about 1000 m²/g.

12. The photothermal magnetic resonance imaging enhancement agent of claim 1, wherein an average pore diameter of the pores is between about 1.5 nm and about 4 nm.

13. The photothermal magnetic resonance imaging enhancement agent of claim 1, wherein the porous substrate comprises a dielectric material selected from the group consisting of silicon dioxide, titanium dioxide, PMMA, polystyrene, dendrimers, and combinations thereof.

14. The photothermal magnetic resonance imaging enhancement agent of claim 13, wherein the porous substrate comprises mesoporous silica.

15. The photothermal magnetic resonance imaging enhancement agent of claim 1, wherein the metallic material comprises a metal selected from the group consisting of coinage metals, noble metals, transition metals, and synthetic metals.

16. The photothermal magnetic resonance imaging enhancement agent of claim 15, wherein the metal comprises gold.

17. The photothermal magnetic resonance imaging enhancement agent of claim 15, wherein the metallic material comprises a metal shell with an average thickness of about 10 nm to about 30 nm.

18. The photothermal magnetic resonance imaging enhancement agent of claim 1, wherein the composite nanoparticle further comprises a coating surrounding the metallic material, wherein the coating comprises molecules that allow one or more of improved nanoparticle stability, facilitating bypassing of an immune system, targeting cells, and increased circulation time.

19. The photothermal magnetic resonance imaging enhancement agent of claim 1, wherein the composite nanoparticle has a surface plasmon resonance between about 800 nm to about 1100 nm.

20. The photothermal magnetic resonance imaging enhancement agent of claim 1, wherein the composite nanoparticle induces a temperature increase of about 20 to about 55° C. upon irradiation with a NIR laser at a laser power of between about 1 W to about 5 W.

21. A method of making a photothermal magnetic resonance imaging enhancement agent, comprising:
synthesizing a dielectric substrate;
baking the dielectric substrate to generate pores within the dielectric substrate;
synthesizing magnetically responsive nanoparticles;
loading the magnetically responsive nanoparticles into the pores of the dielectric substrate so as to form a dielectric inner layer comprising the dielectric substrate and the magnetically responsive nanoparticles;
attaching a plurality of linker molecules to the dielectric substrate;
attaching a metal nanoparticle to each of at least a portion of the linker molecules;
reducing additional metal onto the metal nanoparticle so as to form an outer layer disposed on the dielectric inner layer; and
selecting a condition of the reducing such that the outer layer has a controllable thickness forming a composite nanoparticle.

22. A system for visualizing and inducing hyperthermia in a cell or tissue comprising steps of synthesizing composite nanoparticles, delivering the composite nanoparticles to the cell or tissue, visualizing the composite nanoparticles to ensure site specific delivery, and exposing the composite nanoparticles to infrared radiation under conditions where the composite nanoparticles generate heat upon exposure to the infrared radiation.
